(19) United States
(12) Patent Application Publication
(10) Pub. No.: US 2023/0002312 A1
(43) Pub. Date: Jan. 5, 2023

(54) A CONTINUOUS FLOW PROCESS FOR THE SYNTHESIS OF HYDROXAMIC ACID

(71) Applicant: UPL Limited, Mumbia (IN)

(72) Inventors: Milind Jagannath Pimpale, Mumbai (IN); Prashant Vasant Kini, Mumbai (IN)

(21) Appl. No.: 17/777,364

(22) PCT Filed: Nov. 17, 2020
(86) PCT No.: PCT/IB2020/060803

§ 371 Date: May 17, 2022

(30) Foreign Application Priority Data
Nov. 20, 2019 (IN) 201921047380

(51) Int. Cl. C07C 259/06; B01J 19/00

(52) U.S. Cl. CPC C07C 259/06; B01J 19/0093; B01J 2219/00033; B01J 2219/00894

(57) ABSTRACT

The present invention relates to a process for the synthesis of hydroxamic acids by continuous flow process wherein said process comprising reacting alkyl ester with hydroxyl amine salt in presence of base in a microreactor system and continuously producing hydroxamic acid.

### FIELD OF THE INVENTION

The present invention relates to a continuous flow process for the synthesis of hydroxamic acids. The present invention more particularly relates to synthesis of hydroxamic acids in a microreactor system.

### BACKGROUND OF THE INVENTION

Hydroxamic acids can be represented by the structural formula R₁C(O)N(OH)R₂, where R₁ is typically hydrogen or a hydrocarbon radical such as an alkyl radical, a cycloalkyl radical or an aromatic radical and R₂ can be a hydrogen atom or a hydrocarbon radical such as an aromatic radical or an alkyl radical.

Hydroxamic acids are known to exhibit microbicidal effect and can be employed in practice for controlling undesirable microorganisms. The active compounds are suitable for use as phytoprotective agents, in particular as fungicides. Fungicidal agents in plant protection are employed for combating plasmodiophoromycetes, oomycetes, chytridiomycetes, zygomycetes, ascomycetes, basidiomycetes and deuteromycetes.

Basically, hydroxamic acids have been prepared by different methods, the most common two are; the reaction between acid chloride and hydroxylamine, and the other between esters and hydroxylamine. In the reaction between an ester and hydroxylamine, an alkyl or aryl ester reacts with hydroxylamine in the presence of alkali, the free acid obtained by acidification of cold solution, this reaction takes place in an absolute alcohol and proceeds rapidly at room temperature particularly in presence of an equimolar quantity of sodium alkoxide. In the reaction between acid chloride and hydroxylamine, the N-substituted hydroxylamine is acylated at low temperature diethyl ether medium containing aqueous suspension of sodium hydrogen carbonate.

U.S. Pat. No. 3,922,872 disclose an improved method of making fatty hydroxamates. Hydroxylamine sulfate and the methyl ester of a fatty acid are reacted in the presence of dimethylamine in an anhydrous lower alcohol slurry. The free hydroxamic acids formed are neutralized with dimethylamine or an alkali metal base to yield an ammonium or alkali metal salt. However, the disclosed procedure also employs flammable lower alcohols, such as methanol, ethanol or isopropanol, requiring the filtration of the final hydroxamic product, which is hazardous. Moreover, because of the heterogeneous nature of the reaction, the reaction rate is very slow, e.g., on the order 15 hours in methanol and 5 days in isopropyl alcohol, and the yields are relatively low, i.e., on the order of about 75 percent.

CN103922968A disclosed a process of preparation of hydroxamic acid or hydroxamate. In this process a base is added to a methanol solution of hydroxylamine salt at temperature not more than 45° C. which is then further added to an organic carboxylate, for 2-6 hours at 30˜70° C. After completion of the reaction, system was cooled to below 30° C., sulfuric acid was added to the reaction system, and then methanol was recovered by distillation. The drawback of this process is the lower temperature that increases batch cycle time up to 6 hours. Further, distillation step also requires more cost as compare to processes without solvent.

U.S. Pat. No. 6,288,246 disclosed a process for preparing a molecule containing a hydroxamic acid group, comprising reacting hydroxylamine, or a salt thereof, with a((C₁-C₆)alkyl)₃silyl halide, preferably ((C₁-C₆)alkyl)₃silyl chloride, in the presence of a base, followed by reaction with a carboxylic acid halide containing molecule followed by reaction with an acid, with the proviso that the carboxylic acid halide containing molecule does not contain a hydroxy, primary amine, secondary amine or thiol group. The drawback of this process is long reaction time of 12 hours and also reaction temperature is kept as low as 0° C. to 30° C. that slows down the reaction.

Moreover, the processes disclosed above are batch processes, which can require intermittent introduction of frequently changing raw materials, varying process conditions within the vessel, and different purification methods. Typically, in batch processing, vessels are often idle while waiting for raw materials or undergoing quality control checks and cleaning. Therefore, need exist in the art for simple and rapid process for preparation of hydroxamic acid.

Continuous flow processes allow a constant feed of raw materials to the process vessel and continual product withdrawal. Continuous flow process is very promising recent micro reaction technology, as it offers, as compared to the traditional batch system, a very uniform residence time, much better thermal control, and a lower hold-up, leading to a significant step change in terms of chemical yield and selectivity, and safety. Continuous flow microreactors are now widely used in labs for testing and developing new routes of synthesis. For laboratory and development work, they offer a very small hold-up with a sufficient residence time, leading to a very small use of material for testing, which is of particular interest in the development phase, shortening the time required to make a requested quantity, and when the raw material is expensive. In addition, the small amount of material involved makes reduces safety and environmental risks significantly.

### OBJECTS OF THE INVENTION

An object of the present invention is to provide a process for the synthesis of hydroxamic acids by continuous flow process.

Another object of the present invention is to provide a process for the synthesis of aliphatic hydroxamic acids in a microreactor system.

Yet another object of the invention is to provide a single step continuous flow process for the synthesis of aliphatic hydroxamic acids from lower alkyl esters. Still another object of the invention is to provide a simple and rapid continuous flow process for the synthesis of aliphatic hydroxamic acids in high purity.

### SUMMARY OF THE INVENTION

In an aspect the present invention provides a process for the synthesis of hydroxamic acids by continuous flow process.

In another aspect the present invention provides a process comprising synthesis of hydroxamic acid by reacting alkyl ester with hydroxylamine in presence of base in a microreactor system and continuously producing hydroxamic acid.

In another aspect the present invention is to provides a process for the synthesis of aliphatic hydroxamic acids comprising reacting lower alkyl ester with hydroxylamine in presence of base in a microreactor system and continuously producing hydroxamic acid.

In another aspect of the present invention there is provided a process comprising a continuous flow process for preparation of hydroxamic acids:
charging alkyl ester through a first line of a microreactor unit, in a continuous flow;
charging a solution containing hydroxylamine salt through a second line of the microreactor unit, in a continuous flow;
charging a base solution through a third line of the microreactor unit, in a continuous flow;
reacting alkyl ester, hydroxylamine salt in presence of base in a microreactor to form a product stream of aliphatic hydroxamic acid.

In an aspect of the present invention, a process comprising a continuous flow process for preparation of acetohydroxamic acid:
charging using a first line, in a continuous flow, a solution containing ethyl acetate to a reaction vessel;
charging using a second line, in a continuous flow, a solution containing hydroxylamine salt or the equivalent to a reaction vessel;
charging using a third line, in a continuous flow, a base in the form of solution to a reaction vessel;
permitting reaction of ethyl acetate, hydroxylamine salt or the equivalent and base in the reaction vessel to form acetohydroxamic acid.

In another aspect of the present invention there is provided a system comprising a microreactor unit for producing hydroxamic acid by continuous flow process wherein
charging alkyl ester through a first line of a microreactor unit, in a continuous flow;
charging a solution containing hydroxylamine salt through a second line of the microreactor unit, in a continuous flow;
charging a base solution through a third line of the microreactor unit, in a continuous flow;
reacting alkyl ester, hydroxylamine salt in presence of base in a microreactor to produce a product stream of hydroxamic acid.

### BRIEF DESCRIPTION OF DRAWINGS

The drawings described herein are for illustrative purposes only of selected embodiments and not all possible implementations and are not intended to limit the scope of the present disclosure.

FIG. 1 shows a diagram of a microreactor arrangement with one microreactor vessel for aliphatic hydroxamic acid synthesis.

FIG. 2 shows a diagram of a microreactor arrangement with two microreactor vessels for aliphatic hydroxamic acid synthesis.

FIG. 3 shows a diagram of a microreactor arrangement with one loop reactor and two Plug Flow type microreactor vessels attached adjacent to each other for aliphatic hydroxamic acid synthesis.

### DETAILED DESCRIPTION OF THE INVENTION

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

Broadly, this invention contemplates a process of preparing aliphatic hydroxamic acids from lower alkyl esters which comprises contacting lower alkyl ester from hydroxylamine salts in the presence of a base. The process contemplated by this invention is further explained by the following reaction scheme.

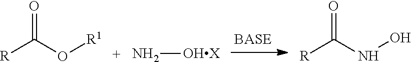

wherein, R represents linear or branched C1-C6 alkyl group, halogenated C1-C6 alkyl group, hydroxy C1-C6 alkyl group, C1-C6 alkoxy C1-C6 alkyl group or C1-C6 cycloalkyl group;

R1 represents linear or branched C1-C6 alkyl group, halogenated C1-C6 alkyl group, hydroxy C1-C6 alkyl group, C1-C6 alkoxy C1-C6 alkyl group or C1-C6 cycloalkyl group;

X represents salts with inorganic bases, salts with organic bases, salts with inorganic acids, salts with organic acids, and salts with basic or acidic amino acids.

Preferable examples of salts with inorganic bases include salts with alkali metals such as sodium, potassium, etc., salts with alkaline earth metals such as calcium, magnesium, etc., and salts with aluminum, ammonium and the like.

preferable examples of salts with organic bases include salts with hydroxylamine, trimethylamine, triethylamine, pyridine, picoline, ethanolamine, diethanolamine, triethanolamine, dicyclohexylamine, N,N-dibenzylethylenediamine and the like. preferable examples of salts with inorganic acids include salts with hydrochloric acid, hydrobromic acid, nitric acid, sulfuric acid, phosphoric acid and the like. preferable examples of salts with organic acids include salts with formic acid, acetic acid, trifluoroacetic acid, fumaric acid, oxalic acid, tartaric acid, maleic acid, citric acid, succinic acid, malic acid, methanesulfonic acid, benzenesulfonic acid, p-toluenesulfonic acid and the like.

Preferable examples of salts with basic amino acids include salts with arginine, lysine, ornithine, etc., and preferable examples of salts with acidic amino acids include salts with aspartic acid, glutamic acid and the like.

The present continuous flow process is beneficial over the traditional batch vessels with following advantages: (i) mass and heat transfer can be significantly improved by decreasing reactor size; (ii) fewer transport limitations can be offered by the feasibility and device flexibility of continuous flow synthesis; (iii) yield and selectivity can be improved due to the precise control of reaction variables such as temperature, pressure and residence time, (iv) scale-up of continuous flow synthesis is readily achieved by simply increasing the number of reactors or their sizes.

The present inventors motivated by these advantages and work out a continuous flow synthesis in a microreactor for the manufacture of hydroxamic acid. The present inventors performed various continuous flow screening experiments to find the residence time and temperature that resulted in the maximum yield and high purity of hydroxamic acid.

In an aspect the present invention provides a process for production of hydroxamic acid comprising mixing alkyl ester with hydroxylamine salt in presence of a base at predetermined conditions of temperature and pressure and flow rate in a microreactor system.

The microreactor used in the process according to the invention may comprise further functional units which exert additional functions in the chemical process regime. The configuration of such functional units is known to a person skilled in microreactor synthesis. For example, microreactor can be selected from the group comprising of Plug Flow Reactor (PFR), Continuous Stirred Tank Reactor (CSTR), Loop reactor, Packed Bed Reactor (PBR) and combinations thereof.

The microreactor system of the present invention can comprise 10 to 100 parallel microreaction systems. Typically, the microreactor systems comprise one or more mixing reactors, one or more reaction reactors, one or more mixing and reaction reactors, one or more heating and cooling element or any combinations thereof, which may be designed in such a way that it is jacketed to maintain temperature and pressure of the reaction vessels in the system.

The present invention has the advantage of short residence time of the material, high selectivity, high yield, less equipment investment, manufacturing cost savings, reduced material consumption, reducing the amount of byproducts. Accordingly, the entire process is technically advanced over the conventional process, continuous, low energy consumption, an efficient and feasible continuous synthesis of aliphatic hydroxamic acid.

Thus, the present invention provides a micro-reactor synthesis for continuous operation for production of hydroxamic acid in high yield and purity.

In accordance with this invention, there is provided a continuous flow process for preparation of aliphatic hydroxamic acids comprising the steps of:
a) charging alkyl ester through a first line of a microreactor unit, in a continuous flow;
b) charging a solution containing hydroxylamine salt through a second line of the microreactor unit, in a continuous flow;
c) charging a base solution through a third line of the microreactor unit, in a continuous flow;
d) reacting alkyl ester, hydroxylamine salt in presence of base in a microreactor to form a product stream of aliphatic hydroxamic acid.

The product stream containing aliphatic hydroxamic acid is then collected in a vessel connected to the microreactor.

A continuous flow process as used herein is not particularly limited, and should be known to a person of ordinary skill in the art. In general, for example and without limitation, a continuous flow process can allow a continuous flow of reactants that can be charged in a reactor, vessel or line, allowing mixing or reaction of the reactants to form products. This is followed by continuous flow (discharge) of the products from the reactor, vessel or line. Thus, a continuous flow process can be considered as a process where reactants are charged or fed into a reactor, vessel or line, while a product is simultaneously removed during part of the reaction process. A continuous flow process can allow a single step or multiple steps to be performed, where each step independently of the other can be a reaction, separation or purification.

The term “alkyl” as used herein refers to the radical of saturated aliphatic groups, including straight-chain alkyl groups, branched—chain alkyl groups, cycloalkyl (alicyclic) groups, alkyl substituted cycloalkyl groups, and cycloalkyl substituted alkyl groups. Examples of alkyl groups include, but are not limited to, methyl, ethyl, propyl, isopropyl, cyclopropyl, butyl, isobutyl, tert-butyl, cyclobutyl, hexyl, cyclochexyl, and the like.

The term “continuous” used herein refers to one or more reagent stream(s) that flow continuously from one reaction step to the next without an intervening isolation or purification step.

The term “line” as used herein is not particularly limited and should be known to a person of skill in the art. In general, a line refers to, for example and without limitation, a tube, conduit or pipe for conveying or transporting fluids. In a continuous flow process, the line can be designed as an inlet and/or outlet to allow charging and/or discharging of fluids, such as reactants or products. In addition, the line (such as, in a reaction mixing line) can be designed to receive reactants and allow mixing and/or reaction of the reactants. Where the line is designed to receive reactants, the size and shape of the line can be adapted to enhance mixing and permit flow of the reactants into the line, minimizing back pressure.

The term “reactor” or “vessel” as used herein are not particularly limited and should be known to a person of skill in the art. In general, a reactor or vessel relates to, for example and without limitation, a container or vat designed to receive chemicals for a chemical process, such as a chemical reaction. In a continuous flow process, the reactor or vessel can be designed to receive continuous charge of the reactants, optionally, a residence time of the reactants within the reactor or vessel, to allow mixing and/or reaction of the reactants to form the products, followed by a continuous discharge of the products. The reactor or vessel can be provided with means, such as, an agitator or baffles to allow mixing of the reactants.

The term “residence time” used herein refers to the time it takes for a molecule in a reagent stream to travel the entire length of a microreactor. The residence time for a reagent stream in a microreactor may depend on the length and width of the microreactor as well as the flow rate of the reagent stream.

The term “solution” as used herein is not particularly limited and should be known to a person of skill in the art. In general, a solution is a homogeneous mixture composed of only one phase. In such a mixture, a solute is a substance dissolved in another substance, known as a solvent. The solvent does the dissolving. The solution more or less takes on the characteristics of the solvent including its phase and the solvent is commonly the major fraction of the mixture. The term solution as used herein can include a mixture having some solids that are not present in solution or insoluble in the solvent, so long as they do not interfere with the overall reaction and process.

In further aspect the present invention provides a system comprising a microreactor unit for producing hydroxamic acid by continuous flow process wherein
charging alkyl ester through a first line of a microreactor unit, in a continuous flow;
charging a solution containing hydroxylamine salt through a second line of the microreactor unit, in a continuous flow;
charging a base solution through a third line of the microreactor unit, in a continuous flow;
reacting alkyl ester, hydroxylamine salt in presence of base in a microreactor to produce a stream of aliphatic hydroxamic acid.

In an embodiment the present invention provides a system comprising a microreactor unit for producing hydroxamic acid by continuous flow process wherein
charging using a first line, in a continuous flow, a solution containing ethyl acetate to a reaction vessel;
charging using a second line, in a continuous flow, a solution containing hydroxylamine salt to a reaction vessel;
charging using a third line, in a continuous flow, suitable base to a reaction vessel;
permitting reaction of ethyl acetate, hydroxylamine salt or the equivalent and base in the reaction vessel to form acetohydroxamic acid.

The process for the production of hydroxamic acid according to the present invention is illustrated in following embodiments, but not limited to, the subsequent description and the figures/drawings referred therein.

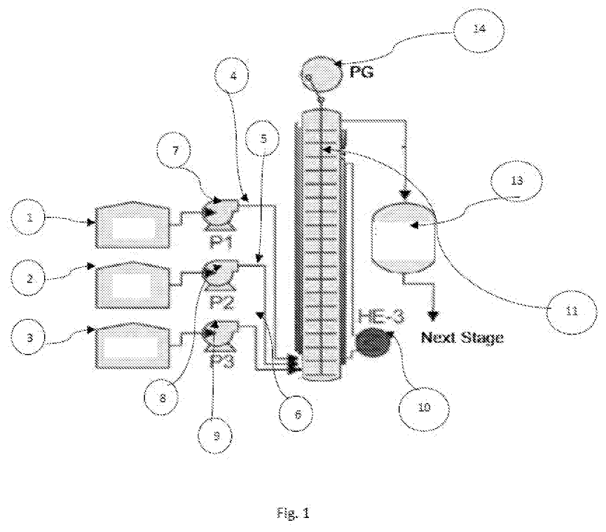

Referring to FIG. 1, a schematic of an exemplary continuous flow reactor for synthesis of hydroxamic acid, the microreactor is a Plug Flow Reactor (PFR) with one reaction vessel (11), having 50 ml capacity. The reaction vessel (11) is designed in such a way that it is jacketed to maintain required temperature and pressure according to conditions of the reaction. Heating elements HE3 (10) is attached to the reaction vessel (11) to provide requisite temperature. Feed containers 1, 2, and 3 are connected to the reaction vessel(11) by tubular components known as mixing lines 4, 5 and 6. Feed containers 1,2 and 3 are connected to the mixing lines 4, 5 and 6 respectively and holds the reactants separately. Pumps 7, 8 and 9 are attached to these mixing lines 4, 5 and 6 such that it drives the reactants contained in the feed containers 1, 2 and 3 to the reaction mixing vessel (11). First mixing line 4, is connected to the reaction vessel (11) via pump (7). Second mixing line 5, is connected to the reaction vessel (11) via pump (8). Third mixing line 6, is connected to the reaction vessel (11) via pump (9). The pressure element is (14) is connected to the reaction vessel (11) to provide pressure adjustment externally. The reaction vessel (11) is connected to collector vessel (13) from where the final product is collected.

According to an embodiment of the present invention, in the continuous flow, a second loop reactor is connected to Plug Flow Reactor such that loop reactor and

Plug Flow Reactor are placed in series as adjacent to each other and are attached via line.

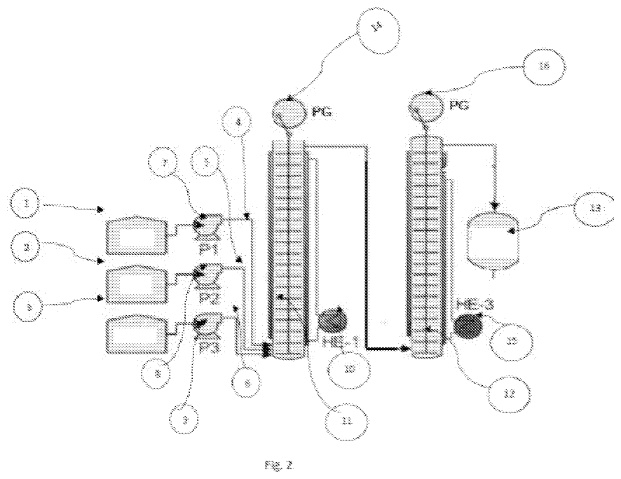

In an embodiment, the microreactor system as represented in FIG. 1 comprises a loop reactor as represented in FIG. 2 and the process for synthesis of hydroxamic acid is carried out according to present invention, as described above.

Referring to FIG. 2, the microreactor described is a Plug Flow Reactor (PFR) with two reaction vessels (11) and (12), having 50 ml each capacity. Therefore, total capacity of PFR microreactor is 100 ml. The reaction vessels (11) and (12) are designed in such a way that they are jacketed to maintain required temperature and pressure according to conditions of the reaction. Heating element HE1 (10) is attached to the reaction vessel (11) and heating element HE3 (15) is attached to the reaction vessel (12) to provide requisite temperature. Feed containers 1, 2, and 3 are connected to the reaction vessel (11) by tubular components known as mixing lines 4, 5 and 6. Feed containers 1,2 and 3 are connected to the mixing lines 4, 5 and 6 respectively and holds the reactants separately. Pumps 7, 8 and 9 are attached to these mixing lines 4, 5 and 6 such that it drives the reactants contained in the feed containers 1, 2 and 3 to the reaction mixing vessel (11). First mixing line 4, is connected to the reaction vessel (11) via pump (7). Second mixing line (5), is connected to the reaction vessel (11) via pump (8). Third mixing line (6), is connected to the reaction vessel (11) via pump (9). The reaction vessel 11 is connected to reaction vessel (12) via extension line (13) to facilitate uniform distribution of reactants in both the reaction vessel (11) and (12) respectively. The pressure element is (14) and (16) are connected to the reaction vessel (11) and reaction vessel (12)respectively to provide pressure adjustment externally. The reaction vessel (12) is connected to collector vessel (13) from where the final product is collected and taken out.

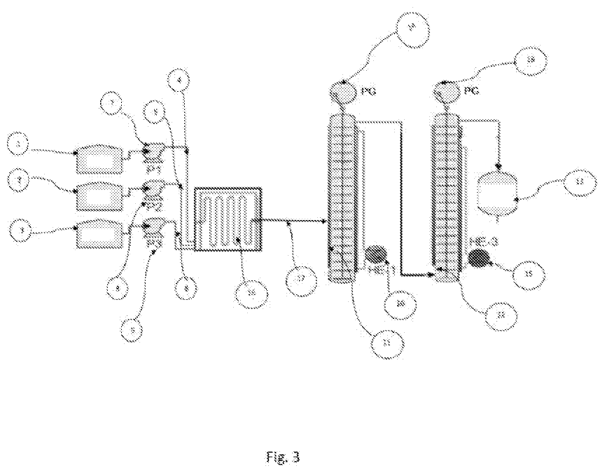

In an embodiment, the microreactor system as represented in FIG. 1 comprises a loop reactor placed prior to Plug Flow Reactor such that the reactants are pre-mixed prior to flowing into Plug Flow Reactor to obtain pre-mix and the pre-mix is then allowed to pass through PFR as represented in FIG. 3 and the process for synthesis of hydroxamic acid is carried out according to present invention, as described above.

Referring to FIG. 3, the microreactor described is a Plug Flow Reactor (PFR) with two reaction vessels (11) and (12), having 50 ml each capacity. Therefore, total capacity of PFR microreactor is 100 ml. The reaction vessels (11) and (12) are designed in such a way that they are jacketed to maintain required temperature and pressure according to conditions of the reaction. Heating element HE1 (10) is attached to the reaction vessel (11) and heating element HE3 (15) is attached to the reaction vessel (12) to provide requisite temperature. Feed containers 1, 2, and 3 are connected to loop reactor 6 tubular components known as mixing lines 4, 5 and 6. Feed containers 1,2 and 3 are connected to the mixing lines 4, 5 and 6 respectively and holds the reactants separately. Pumps 7, 8 and 9 are attached to these mixing lines 4, 5 and 6 such that it drives the reactants contained in the feed containers 1, 2 and 3 to the loop reactor 16. First mixing line (4), is connected to the loop reactor (16)via pump (7). Second mixing line (5), is connected to the loop reactor (16)via pump (8). Third mixing line (6), is connected to the loop reactor via pump (9). Loop reactor (16) receives reactants via mixing lines 4, 5 and 6 and facilitate pre-mixing of reactants. Pre-mix of reactants then allowed to pass to the reaction mixing vessel (11) via connector pipe (17). The reaction vessel (11) is connected to reaction vessel (12) via extension line (13) to facilitate uniform distribution of reactants in both the reaction vessel (11) and (12) respectively. The pressure element 14 and 16 are connected to the reaction vessel (11) and reaction vessel (12)respectively to provide pressure adjustment externally. The reaction vessel (12) is connected to collector vessel (13) from where the final product is collected and taken out.

In accordance with this invention, there is provided a continuous flow process for preparation of aliphatic hydroxamic acids comprising the steps of:
a) charging alkyl ester through a first line of a microreactor unit, in a continuous flow;
b) charging a solution containing hydroxylamine salt through a second line of the microreactor unit, in a continuous flow;
c) charging a base solution through a third line of the microreactor unit, in a continuous flow;
d) reacting alkyl ester, hydroxylamine salt in presence of base in a microreactor to form a product stream of aliphatic hydroxamic acid.

The product stream containing aliphatic hydroxamic acid is then collected in a vessel connected to the microreactor.

According to an embodiment of the present invention, a continuous flow process for the synthesis of aliphatic hydroxamic acids is depicted in Scheme showed above, wherein lower alkyl esters for the synthesis of aliphatic hydroxamic acids in the continuous flow process are selected from the group comprising of methyl acetate, ethyl acetate, propyl acetate, butyl acetate, amyl acetate, hexyl acetate, heptyl acetate, octyl acetate, 3-methyl butyl acetate, propan-2-yl-acetate, 2-methylpropyl acetate, ethyl butanoate.

In an embodiment of the present invention, lower alkyl esters for the synthesis of aliphatic hydroxamic acids in the continuous flow process are selected from ethyl acetate and methyl acetate.

In a preferred embodiment of the present invention, lower alkyl esters for the synthesis of aliphatic hydroxamic acids in the continuous flow process is ethyl acetate.

According to an embodiment of the present invention, hydroxylamine salts for the synthesis of aliphatic hydroxamic acids in the continuous flow process are selected from the group comprising of hydroxylammonium nitrate (also referred to as HAN), hydroxylammonium sulfate (also referred to as HAS), hydroxylammonium phosphate, hydroxylammonium chloride, hydroxylammonium oxalate, hydroxylammonium citrate and the like.

In an embodiment of the present invention, hydroxylamine salts for the synthesis of aliphatic hydroxamic acids in the continuous flow process are selected from hydroxylammonium sulfate and hydroxylammonium chloride.

In a preferred embodiment of the present invention, hydroxylamine salts for the synthesis of aliphatic hydroxamic acids in the continuous flow process is hydroxylammonium sulfate.

According to an embodiment of the present invention, suitable base for the synthesis of aliphatic hydroxamic acids in the continuous flow process are selected from the group comprising of lithium hydroxide, sodium hydroxide, potassium hydroxide, rubidium hydroxide, cesium hydroxide, magnesium hydroxide, calcium hydroxide, strontium hydroxide, barium hydroxide.

In an embodiment of the present invention, suitable base for the synthesis of aliphatic hydroxamic acids in the continuous flow process are selected from sodium hydroxide and sodium chloride.

In an embodiment of the present invention, the continuous flow process for the synthesis of aliphatic hydroxamic acids is carried out in microreactors selected from the group comprising of Plug Flow Reactor (PFR), Continuous Stirred Tank Reactor (CSTR), Loop reactor, Packed Bed Reactor (PBR) and combinations thereof.

In an embodiment of the present invention, the continuous flow process for the synthesis of aliphatic hydroxamic acids is carried out in Plug Flow Reactor (PFR). In an embodiment of the present invention, the continuous flow process for the synthesis of aliphatic hydroxamic acids is carried out in Loop Reactor.

In an embodiment of the present invention, the continuous flow process for the synthesis of aliphatic hydroxamic acids is carried out by combining Loop Reactor and Plug Flow Reactor (PFR) in series such that reactants are first allowed to get mixed in Loop Reactor to obtain pre-mix and the pre-mix is then allowed to pass through PFR.

According to an embodiment of the present invention, flow rate of reactants flowing from first line varies from 1 ml/min to 20 ml/min in a reactor upto 100 ml capacity.

According to an embodiment of the present invention, flow rate of reactants flowing from second line varies from 1 ml/min to 20 ml/min in a reactor upto 100 ml capacity.

According to an embodiment of the present invention, flow rate of reactants flowing from third line varies from 1 ml/min to 20 ml/min in a reactor upto 100 ml capacity.

According to an embodiment of the present invention, flow rate of reactants from first, second and third line of microreactor may vary on the basis of desired output volume of aliphatic hydroxamic acid.

According to an embodiment of the present invention, the volume of microreactors for carrying out the continuous flow process for the synthesis of aliphatic hydroxamic acid at laboratory scale are selected from various capacity range of 1 ml, 10 ml, 50 ml, 100 ml and the like based on desired output volume of aliphatic hydroxamic acid.

According to an embodiment of the present invention, volume of microreactors for carrying out the continuous flow process for the synthesis of aliphatic hydroxamic acid at commercial scale are selected from various capacity range of 1 L, 10 L, 50 L, 100 L, 5000 L, 50000 L and can be more which can be based on desired output volume of aliphatic hydroxamic acid.

According to some embodiments, synthesis of aliphatic hydroxamic acid occurs in shorter reaction time, relative to known methods.

According to an embodiment of the present invention, residence time of reactants in the reaction vessel to synthesize aliphatic hydroxamic acid with at least 90% yield is 1 hour or less.

According to an embodiment of the present invention, residence time of reactants in the reaction vessel to synthesize aliphatic hydroxamic acid with at least 99% yield is 1 hour or less.

In some embodiments, residence time of reactants in the reaction vessel to synthesize aliphatic hydroxamic acid may be about 1 hours or less, about 30 min or less or less, or, in some cases, about 20 min or less.

According to one preferred embodiment of the present invention, advantageously the residence time of reactants in the reaction vessel to synthesize aliphatic hydroxamic acid may be about 5 minutes or less.

According to one preferred embodiment of the present invention, residence time is about 60 seconds.

According to one preferred embodiment of the present invention, residence time is about 30 seconds.

Without wishing to be bound by theory, such residence times may be attributed to increase in the rate of a chemical reaction within a microreactor, relative to other processes (for example batch processes), due to rapid mass and heat transfer, high temperatures, and high pressures attainable within a microreactor, as described more fully below.

In one embodiment the process for synthesis of hydroxamic acid comprises reaction of hydroxylamine sulfate with lower alkyl esters in presence of base in a microreactor at a predetermined condition of temperature, pressure and flow rate of reactants to produce hydroxamic acid in high yield and purity.

The present process in the microreactor may be carried out at a temperature from about 50 to about 120° C. and a pressure from about lto about 10 bar.

In a preferred embodiment of the present invention, the reaction vessel is maintained from about 2 to about 5 bar pressure for the synthesis of aliphatic hydroxamic acid.

According to an embodiment of the present invention, temperature of the reaction vessel is about 100 to about 120° C. or less to synthesize aliphatic hydroxamic acid in a continuous flow.

According to an embodiment of the present invention, temperature of the reaction vessel is about 100° C. or less, preferably about 80° C. or less, about preferably about 50° C. or less to synthesize hydroxamic acid in a continuous flow.

According to an embodiment of the present invention, flow rate of ethyl acetate flowing from first line varies from 1 ml/min to 20 ml/min in a reactor upto 100 ml capacity.

According to an embodiment of the present invention, flow rate of hydroxylamine salt solution flowing from second line varies from 1 ml/min to 20 ml/min in a reactor upto 100 ml capacity.

According to an embodiment of the present invention, flow rate of base flowing from third line varies from 1 ml/min to 20 ml/min in a reactor upto 100 ml capacity.

In an aspect of the present invention, a continuous flow process for preparation of acetohydroxamic acid comprises steps of:
a) charging ethyl acetate through a first line of a microreactor unit, in a continuous flow;
b) charging a solution containing hydroxylamine salt through a second line of the microreactor unit, in a continuous flow;
c) charging a base solution through a third line of the microreactor unit, in a continuous flow;
d) reacting alkyl ester, hydroxylamine salt in presence of base in a microreactor to form a product stream of acetohydroxamic acid.

The product stream containing acetohydroxamic acid is then collected in a vessel from the microreactor.

According to an embodiment, the residence time for the synthesis of acetohydroxamic acid in continuous flow process is from about 30 sec to 5 minutes.

According to an embodiment of the present invention, the reaction vessel is maintained from about 2 to about 5 bar pressure for the synthesis of acetohydroxamic acid.

According to another embodiment of the present invention, the temperature of reaction vessel is kept below 90° C.

The feed streams of hydroxylamine salt:alkyl ester:base can be supplied to the microreactor in a stoichiometric ratio of 1:1:1

The feed streams of hydroxylamine salt:alkyl ester:base can be supplied to the microreactor in a stoichiometric ratio of 1:3:3.

The feed streams of hydroxylamine salt:alkyl ester:base can be supplied to the microreactor in a stoichiometric ratio of 1:5:5.

In one embodiment the flow rate is maintained throughout the process in a microreactor so that stoichiometric ratio of hydroxylamine sulfate:ethyl acetate:sodium hydroxide is in the range of 1:3:3 to produce hydroxamic acid.

The process of the present invention provides hydroxamic acid with a yield of at least 90%.

The process of the present invention provides hydroxamic acid with a yield of at least 95%.

The process of the present invention provides hydroxamic acid with a yield of at least 99%.

The process of the present invention provides hydroxamic acid with a purity of at least 90%.

The process of the present invention provides hydroxamic acid with a purity of at least 95%.

The process of the present invention provides hydroxamic acid with a purity of at least 99%.

The process of the present invention provides hydroxamic acid with an high yield of at least 99% and high purity of more than 95%, preferably more than 98%.

Accordingly, hydroxamic acid produced according to the present invention has purity of about 98.5%.

According to an embodiment of the present invention, in the continuous flow, a loop reactor is attached prior to Plug Flow Reactor such that loop reactor and Plug Flow Reactor are placed in series as adjacent to each other and are attached via line.

According to an embodiment of the present invention, loop reactor receives alkyl acetate, hydroxylamine salt and base from first line, second line and third line respectively and forms a pre-mix which is then passed through plug flow reactor to form aliphatic hydroxamic acid.

According to an embodiment of the present invention, flow rate of ethyl acetate flowing from first line to the loop reactor varies from 1 ml/min to 10 ml/min in a reactor having 20 ml capacity.

According to an embodiment of the present invention, flow rate of hydroxylamine salt solution flowing from second line to the loop reactor varies from 1 ml/min to 20 ml/min in a reactor having 30 ml capacity.

According to an embodiment of the present invention, flow rate of base flowing from third line to the loop reactor varies from 1 ml/min to 10 ml/min in a reactor having 30 ml capacity.

According to an embodiment of the present invention, output rate of pre-mix from loop reactor of 30 ml capacity is from about 5 ml/min to about 30 ml/min.

According to an embodiment, the residence time in loop reactor for the synthesis of acetohydroxamic acid in continuous flow process is from about 10 sec to 2 min.

According to another embodiment of the present invention, the temperature of loop reactor is kept below 60° C.

According to another embodiment of the present invention, the reaction in the loop reactor is operated at room temperature.

According to an embodiment of the present invention, the aliphatic hydroxamic acid synthesized in continuous flow process according to the present invention may be used as an intermediate to prepare a cyclohexanone herbicide, particularly for preparing clethodim.

According to an embodiment of the present invention, the aliphatic hydroxamic acid synthesized in continuous flow may be used in synthesizing various chemical, pharmaceutical and agrochemical compounds.

Although the subject matter has been described in considerable detail with reference to certain preferred embodiments thereof, other embodiments are possible. As such, the spirit and scope of the disclosure should not be limited to the description of the preferred embodiment contained therein.

### Advantages of the Present Invention

The present continuous-flow process is simple, fast, high efficiency and easy operation.

The present continuous flow process involves continuous production of hydroxamic acid in a reactor of micro-sized thereby making the material mixing and mass transfer easy and industrially feasible.

The process is continuously carried out by continuously adding fresh reactants without interruption i.e. continuously flowing throughout the process for production of desired product.

Advantageously, the reaction time of the process can be brought down to within 30 second to 5 minutes by present process thereby reducing both cost and operating step of the process.

The following are provided as specific embodiments of the present invention. Other modifications of this invention will be readily apparent to those skilled in the art. Such modifications are understood to be within the scope of this invention.

The invention is illustrated by the following Examples which however do not limit the invention.

### Example 1

Three-line Plug Flow Reactor (PFR) was used to perform continuous reaction. Ethyl acetate (88.11 g) was fed to the reactor by first dosing line at the rate of 7.61 ml/min, 30% solution of Hydroxylamine sulfate (169 gm of hydroxylamine sulfate in to 395 gm of water) was fed to the reactor by second dosing line at the rate of 16.5 ml/min, 30% solution of NaOH (42 gm of sodium hydroxide in to 92.5 gm of water) was fed to the reactor by third line at the rate of 9.2 ml/min. The flow rate is adjusted to maintain stoichiometric ratio of hydroxylamine sulfate:ethyl acetate:sodium hydroxide to about 1:2.15:2.6. All the three dosing lines discharge their contents in the reaction vessel which was maintained at 90° C. to form acetohydroxamic acid within residence time of 3 min. The results of the reaction setup were highlighted in Table 1. Samples were analysed by HPLC (HPLC purity 97%).

### Example 2

Three-line PFR reactor was used to perform continuous reaction. Ethyl acetate (88.11 g) was fed to the reactor by first dosing line at the rate of 7.0 ml/min, 30% solution of Hydroxylamine Hydrochloride (71 gm of hydroxylamine hydrochloride in to 161 gm of water) was fed to the reactor by second dosing line at the rate of 12.64 ml/min, 30% solution of NaOH (42 gm of sodium hydroxide in to 92.5 gm of water) was fed to the reactor by third line at the rate of 13.42 ml/min. The flow rate is adjusted to maintain stoichiometric ratio of hydroxylamine Hydrochloride:ethyl acetate:Sodium hydroxide to 1:1.15:2.0. All the three dosing lines discharge their contents in the reaction vessel which was maintained at 90° C. Without modifying the existing conditions, the desired product, acetohydroxamic acid was formed within residence time of 3 min. The results of the reaction setup were highlighted in Table 1. Samples were analysed by HPLC (HPLC purity 96%).

### Example-3

Three-line PFR was used to perform continuous reaction. Ethyl acetate (88.11 gm) was fed to the reactor by first dosing line at the rate of 7.4 ml/min, 30% solution of Hydroxylamine sulfate (169 gm of hydroxylamine sulfate in to 395 gm of water) was fed to the reactor by second dosing line at the rate of 15.6 ml/min, 30% solution of NaOH (42 gm of sodium hydroxide in to 92.5 gm of water) was fed to the reactor by third line at the rate of 10.38 ml/min. The flow rate is adjusted to maintain stoichiometric ratio of hydroxylamine sulfate:ethyl acetate:Sodium hydroxide to 1:2.2:3. All the three dosing lines discharge their contents in the reaction vessel which was maintained at 90° C. Without modifying the existing conditions, the desired product, acetohydroxamic acid was formed within residence time of 3 min. The results of the reaction setup were highlighted in Table 1. Samples were analysed by HPLC (HPLC purity 98%).

### Example-4

Three-line PFR was used to perform continuous reaction. Ethyl acetate was fed to the reactor by first dosing line at the rate of 7.4 ml/min, 30% solution of Hydroxylamine sulfate was fed to the reactor by second dosing line at the rate of 15.6 ml/min, 30% solution of NaOH was fed to the reactor by third line at the rate of 10.38 ml/min. The flow rate is adjusted to maintain stoichiometric ratio of hydroxylamine sulfate:ethyl acetate:Sodium hydroxide to 1:2.2:3. All the three dosing lines discharge their contents in the reaction vessel which was maintained at 90° C. Without modifying the existing conditions, the desired product, acetohydroxamic acid was formed within residence time of 3 min. The results of the reaction setup were highlighted in Table 1. Samples were analysed by HPLC (HPLC purity 98%).

### Example-5

Three-line PFR was used to perform continuous reaction. Ethyl acetate was fed to the reactor by first dosing line at the rate of 5.1 ml/min, 35% solution of Hydroxylamine sulfate was fed to the reactor by second dosing line at the rate of 9.08 ml/min, 35% solution of NaOH was fed to the reactor by third line at the rate of 5.9 ml/min. The flow rate is adjusted to maintain stoichiometric ratio of hydroxylamine sulfate:ethyl acetate:Sodium hydroxide to 1:2.4:3.0 All the three dosing lines discharge their contents in the loop reactor connected prior to reaction vessel of PFR via tube. A pre-mix was obtained by mixing of all reactants in the loop reactor at temperature 50° C. The pre-mix was then fed via tube to reaction vessel of PFR maintained at 50° C. Without modifying the existing conditions, the desired product, acetohydroxamic acid was formed within total residence time of 5.9 min. The results of the reaction setup were highlighted in Table 1. Samples were analysed by HPLC (HPLC purity 98%).

Study of Process Parameters:

To evaluate the effect of flow rate, reactor volume and temperature on the yield of aliphatic hydroxamic acids, various experiments were conducted in Plug Flow Rate (PFR) microreactor by varying flow rate, reactor volume and temperature. Upon optimization it was found that flow rate, reactor volume and temperature played a critical role in the synthesis of hydroxamic acids. The process parameters and results were summarised in Table 1.

TABLE 1

| | | Flow Rates (ml/min) | | | | | | | | |
|---|---|---|---|---|---|---|---|---|---|---|
| | | Flow | Flow | Flow | | | | | | |
| | Reactor | rate | rate | rate | | Mole ratio Alkyl | Reaction | | Purity | |
| | Volume | of 1^{st} | of 2^{nd} | of 3^{rd} | Residencetime | Ester:Hydroxyl | Temp | Pressure | % | |
| Reactor | (ml) | line | line | line | (min) | amine salt:Base | (° C.) | (Bar) | Area | Yield |
| PFR | 50 | 2.38 | 4.45 | 3.16 | 5 | 1:2.04:2.04 | 50 | 2-3 | 85.57 | 90% |
| PFR | 50 | 2.38 | 4.45 | 3.16 | 5 | 1:2.04:2.04 | 60 | 2-3 | 86.02 | 91% |
| PFR | 50 | 2.38 | 4.45 | 3.16 | 5 | 1:2.04:2.04 | 70 | 2-3 | 88.10 | 93% |
| PFR | 100 | 7.61 | 16.5 | 9.2 | 3 | 1:2.15:2.5 | 80 | 4-5 | 95.10 | 96% |
| PFR | 100 | 7.61 | 16.5 | 9.2 | 3 | 1:2.15:2.5 | 90 | 4-5 | 97.02 | 98% |

Tests were performed on two reactor vessels, 50 ml and 100 ml capacity respectively. Repetitive batches were taken in these reactors by varying process parameters, such as flow rates, residence time, reaction temperature and pressure. The temperature of the reaction was varied from 50° C. to 90° C. and effect of varying temperature was evaluated against yield of aliphatic hydroxamic acids. Optimum pressure required to conduct the reaction was varied between 2-5 bar. It was found that yield upto 98% can be achieved when reaction is conducted at 90° C. and pressure is set upto 4-5 bar in a continuous flow microreactors. It is also observed that the amount of certain impurities formed during synthesis of aliphatic hydroxamic acid reduced drastically, leading to high purity and high yield of the aliphatic hydroxamic acid synthesized in continuous flow system according to the present invention. Inventors of the present invention thus successfully prepared aliphatic hydroxamic acids from lower alkyl esters in a continuous flow. The process described above can synthesize aliphatic hydroxamic acid very quickly under controlled conditions.

1. A process for production of hydroxamic acid comprising reacting an alkyl ester with hydroxylamine salt in presence of a base in a microreactor system and continuously producing hydroxamic acid 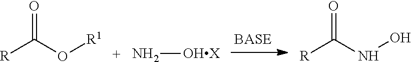
wherein,
R represents a linear or branched C1-C6 alkyl group, halogenated C1-C6 alkyl group, hydroxy C1-C6 alkyl group, C1-C6 alkoxy C1-C6 alkyl group, or C1-C6 cycloalkyl group;
R¹ represents a linear or branched C1-C6 alkyl group, halogenated C1-C6 alkyl group, hydroxy C1-C6 alkyl group, C1-C6 alkoxy C1-C6 alkyl group, or C1-C6 cycloalkyl group; and
X represents a salts with and inorganic bases, a salts with an organic bases, a salts with an inorganic acids, a salts with an organic acids, or a salts with a basic or acidic amino acids.

2. The process as claimed in claim 1, wherein said hydroxamic acid is an aliphatic hydroxamic acids.

3. The process as claimed in claim 1, wherein said hydroxamic acid is acetohydroxamic acid.

4. The process as claimed in claim 1, wherein said alkyl ester is a lower alkyl ester selected from the group consisting of methyl acetate, ethyl acetate, propyl acetate, butyl acetate, amyl acetate, hexyl acetate, heptyl acetate, octyl acetate, 3-methyl butyl acetate, propan-2-yl-acetate, 2-methylpropyl acetate, and ethyl butanoate.

5. The process as claimed in claim 1, wherein said hydroxylamine salt is a salt of hydroxylamine with an inorganic bases, an organic bases, an inorganic acids, an organic acids, or a salts with a basic or acidic amino acids.

6. The process as claimed in claim 1, wherein said hydroxylamine salt is selected from the group consisting of hydroxylammonium nitrate (HAN), hydroxylammonium sulfate (HAS), hydroxylammonium phosphate, hydroxylammonium chloride, hydroxylammonium oxalate, and hydroxylammonium citrate.

7. The process as claimed in claim 1, wherein said base is selected from the group consisting of lithium hydroxide, sodium hydroxide, potassium hydroxide, rubidium hydroxide, cesium hydroxide, magnesium hydroxide, calcium hydroxide, strontium hydroxide, and barium hydroxide.

8. The process as claimed in claim 1, wherein the process is a continuous flow process carried out in microreactor selected from the group consisting of a plug flow reactor (PFR), a continuous stirred tank reactor (CSTR), a loop reactor, a packed bed reactor (PBR) or a and combinations thereof.

9. The process as claimed in claim 1, wherein a residence time of reactants in the process is from about 30 seconds to 1 hour.

10. The process as claimed in claim 1, wherein a temperature of the reaction is from about 50 to about 120° C.

11. The process as claimed in claim 1, wherein a pressure of the reaction vessel is from about 1 to about 10 bar.

12. The process as claimed in claim 1, wherein the hydroxamic acid produced has a is having purity of at least 95%.

13. A continuous flow process for synthesis of hydroxamic acid—comprising:
charging an alkyl ester through a first line of a microreactor unit, in a continuous flow;
charging a solution containing a hydroxylamine salt through a second line of the microreactor unit, in a continuous flow;
charging a base solution through a third line of the microreactor unit, in a continuous flow; and
Reacting the alkyl ester and hydroxylamine salt in the presence of the base in the microreactor unit to form a product stream of aliphatic hydroxamic acid.

14. The process as claimed in claim 13, wherein said process comprises steps of:
charging using the first line, in a continuous flow, a solution containing ethyl acetate to the reaction vessel;
charging using the second line, in a continuous flow, the solution containing the hydroxylamine salt to a reaction vessel;
charging using the third line, in a continuous flow, the base solution to the reaction vessel; and
permitting reaction of the ethyl acetate, hydroxylamine salt or the equivalent and base in the reaction vessel to form acetohydroxamic acid.

15. A system comprising a microreactor unit for producing hydroxamic acid by a continuous flow process as defined in claim 13.
